(19) United States
(12) Patent Application Publication
(10) Pub. No.: US 2004/0137086 A1
(43) Pub. Date: Jul. 15, 2004

(54) Method of altering weight perception

(76) Inventor: Alan R. Hirsch, Riverwoods, IL (US)

Correspondence Address: WHYTE HIRSCHBOECK DUDEK S C, 555 EAST WELLS STREET, SUITE 1900, MILWAUKEE, WI, 53202, US

(21) Appl. No.: 10/690,791

(22) Filed: Oct. 22, 2003

Related U.S. Application Data
(60) Provisional application No. 60/420,288, filed on Oct. 22, 2002.

(51) Int. Cl. A61K 35/78

(52) U.S. Cl. 424/725

(57) ABSTRACT

Methods and compositions for altering perception of weight are disclosed.

### CROSS-REFERENCE TO RELATED APPLICATION

This application claims the benefit of U.S. Provisional Application Serial No. 60/420,288, filed Oct. 22, 2002.

### FIELD OF THE INVENTION

The present invention relates to altering perception of weight by the administration of odorants.

### BACKGROUND OF THE INVENTION

An epidemic of obesity exists in America. One-third of the American population is substantially above their range of desirable body weight. Men's perception of what a woman's attractive weight should be, is diametrically opposed to this. Body mass index is inversely proportional to perceived body attractiveness. (D. M. Garner et al., Cultural Expectations of Thinness in Women, Psychological Reports 47:483-491 (1980); M. Tovee et al., Visual Cues to Female Physical Attractiveness, Proceedings of the Royal Society London (Series B): Biological Sciences 266:211-218 (1999)). This is reflected by Playboy centerfolds and Miss America pageant contestants who have become progressively thinner. Weight, except at the extreme low end, is a measure of attractiveness.

In light of this, the discrepancy that a thinner body image is perceived as being more attractive while the baseline population weight average increases is even more detrimental. This paradox results in social distress and body image dissatisfaction of those who are not underweight. Of 3,193 overweight subjects queried 76% perceived themselves as unattractive or ugly, 52.9% felt dissatisfied with their lives, and 29.7% avoided others because of eating problems. (A. R. Hirsch and R. Gomez, Weight Reduction Through Inhalation of Odorants, Journal of Neurological and Orthopedic Medicine and Surgery 16:26-31 (1995)). Furthermore, poor body image has been directly correlated with impaired social interaction.

Beyond self-perception, a woman's understanding of how others perceive her attractiveness affects social intercourse. Even if she is not overweight, nor perceives herself as such, believing others view her as heavy can affect social interaction. Since women are aware of the adverse social consequences of being considered obese, they attempt to project the visual illusion of thinness. Use of visual cues to produce the illusion of reduced size is demonstrated in the Mueler-Lyer illusion (arrows in vs. arrows out) as well as in the Poggendorf illusion (the disappearance of a line behind a solid figure). (R. A. Baron, In: Psychology, 4th ed., pp. 118-119, Allyn and Bacon, Boston (1998)). Visual illusions are also illustrated in the fashion industry. Visual techniques perceived to conceal, reduce or alternately exaggerate physical attributes are often applied to garments. Patterns with vertical lines, solid black clothing, distracting accessories, and use of contouring garments (i.e. corsets, girdles) are a few of the methods, which are used to emote a thinner image.

These same techniques extend beyond fashion to personal grooming. In one respect, makeup is applied to create or enhance contours in the face to aesthetically enhance face shape. In another respect, makeup is applied in an exaggerated fashion to draw attention to the face and away from regions of the body that one feels makes her appear more corpulent. Likewise, hair is styled and colored to be the focus of attention. Even manipulation of the body itself is used to achieve a slimmer appearance. Muscle activity is initiated; gluteus maximii contraction reduces buttock size and extension of erector spinai reduces girth. Intentional body positioning is also used to disguise unwanted weight. Crossing legs to provide contour and crossing arms to hide panniculus are other techniques.

While all of the above techniques involve visual stimuli, illusions are not limited to this sensory sphere. Variation in temperature and varying frequency of sound can affect the level of pleasure and arousal a person experiences. Illusions effecting perception of other sensory modalities include that of audition, touch, and temperature. One sensory stimulus may even effect perception in a different sensory modality. For instance, auditory stimuli can influence perception of visual stimuli. Odors have been demonstrated to affect perceived quality of inanimate objects, first impressions of others, and perception of size of external space.

Although various methods and treatments have been described to affect perception of body weight, none of these methods have been fully effective. Therefore, It would be useful to provide a method of altering perception of body weight by the use of odorants. It would be useful to provide a means to enable a person suffering from a weight disorder to increase the feeling of well being. It would also be useful to provide a means to enable a person suffering from an eating disorder to better deal with the disorder.

### SUMMARY OF THE INVENTION

The present invention relates to methods of using odorants to alter perception of human body weight. It has been surprisingly found that certain odorants function similar to visual stimuli, to influence perception of attractiveness reflected through estimation of body weight.

The present invention is directed to a method for enhancing the perception of reduced body weight of an individual through the delivery of an odorant or odorant mixture for inhalation. In particular, the method involves delivering an effective amount of an odorant or odorant mixture, preferably one with positive hedonics, to an individual for inhaling for an effective period of time to cause the individual's perception of another person's body weight to be lower than the actual body weight measurement, preferably lower by at least about 10% or more. In a preferred embodiment, the method involves the application of a hedonically positive mixture of floral and spice odorants.

The method can be effectively practiced to alleviate and/or reduce anxiety and/or phobias of an individual that are related to a negative perception of their body weight, i.e., negative feelings of being overweight, and increase their feelings of well being and attractiveness. In particular, the method can be utilized practiced on a “viewing” individual (who inhales the odorant/mixture) to affect their perception of the body weight of another individual who is overweight, particularly an individual who ranges from obese to morbidly obese, to be less than the actual weight resulting in a positive perception of the overweight individual. The altered perception of the viewing individual can result in an anxiety reducing or calming effect on the overweight individual and increase their feelings of well being associated with a positive perception of their body weight.

In another embodiment, the method can be utilized in treating a person diagnosed with an eating disorder such as bulimia nervosa, binge eating disorder and anorexia nervosa, by administering an effective amount of one or more hedonically positive odorants to the person for inhalation, such that the person perceives their body weight to be less than their actual body weight. The method can be combined with behavior modification therapy.

In another embodiment, the method can be utilized for treating a person diagnosed with a psychological disorder associated with perception of body weight, for example, body dysmorphic disorder, a social phobia and pathological shyness, by administering one or more hedonically positive odorants to the person for inhaling such that the person perceives their body weight to be lower than their actual body weight. The method can also be combined with behavior modification therapy.

In yet another embodiment, the method can be utilized to affect the perception of a viewing individual (who inhales the odorant or odorant mixture) of the body weight of an individual suffering from an eating disorder (e.g., anorexia, bulimia, etc.), and/or of an individual suffering from a psychological disorder (e.g., body dysmorphic disorder, etc.) to generate a positive effect by the viewing individual of the individual suffering from the disorder. This altered perception can result in an anxiety reducing or calming effect on the individual suffering from the disorder and increase their feelings of well being associated with a positive perception of their body weight.

The method of the invention can also be used as an adjuvant or aid to assist in treatments for weight problems including obesity, and treatments to rehabilitate patients diagnosed with eating disorders or psychiatric disorders by reducing anxiety associated with feelings of being overweight, and/or enhancing a positive perception of body weight and/or size.

### DETAILED DESCRIPTION OF THE INVENTION

According to the method, an odorant or odorant mixture is administered to a subject for sniffing and inhalation into the nasal passageway, to deliver an amount of the odorant or odorant mixture effective to alter the subject's perception of weight. The odorant or odorant mixture can be formulated to cause an alteration in perception of the person inhaling the mixture of the body weight of another individual. Such an effect can be assessed and measured subjectively by interviewing and questioning the individual about their perception of body weight of another individual before and after the administration of the odorant, and assessing their response. Preferably, the odorant or odorant mixture that is administered is an aromatic substance to which the inhaling individual displays a positive hedonic response.

As used herein, the term “odorant” refers to an odor-causing chemical compound or mixture of compounds that, when delivered in a gaseous or aerosol medium, can stimulate olfactory and/or trigeminal chemoreceptors in the nasal cavity and cause a physiological or psychological response. A hedonically positive odorant or odorant mixture is one to which the individual has a pleasant or positive reaction to its scent.

In a preferred embodiment, the subject individual is presented with a suprathreshold concentration (e.g., about 25-55 decismel units) of the odorant or odorant mixture that is not so high as to become an irritant (trigeminal), and generally inhales the odorant for about three seconds to about one minute, preferably about 20 seconds. Preferably, the level or concentration of the odorant or odorant mixture and/or mode of administering the composition is sufficient to overcome competing or conflicting ambient odors that may act to nullify its effect.

It is preferred that the effect of the odorant/odorant mixture on the perception of a normosmic person who finds the odorant or odorant mixture to be hedonically positive provides a change in perception of at least about 7% reduction to up to about 10% or greater reduction in perceived body weight. For normosmics who consider the odorant or odorant mixture to be hedonically negative, the result is preferably a change in perception of an at least about 3% reduction to up to an about 5% or greater reduction in perceived body weight.

According to the invention, it was found that the administration of a mixture of a floral odorant and a spice odorant will significantly alter the perception of a person inhaling the odorant mixture of the body weight of a person who is visibly overweight, particularly an obese or morbidly obese person. In particular, it was found that the inhalation of a hedonically positive floral-spice odorant mixture can significantly alter the perception of a person such that the perceived body weight of an overweight person is lower than the person's actual body weight by up to about 10%.

The odorant or odorant mixture is provided as a single essential odorant or a formulated blend of the essential odorants to cause the desired effect, and eliminates odorants that compete with or mask the effective odorant(s). The odorant or odorant mixture can be administered in combination with an odorless carrier such as mineral oil or water, and odorless additives such as preservatives and the like. Preferably, the odorant or odorant mixture provides a hedonically positive response by the inhaling individual.

The preferred floral-spice odorant mixture is a formulation that essentially comprises a hedonically positive blend of floral and spice odorants and eliminates odorants that compete with the floral and spice accords or notes to provide a full effect on the individual inhaling the odorant mixture. Examples of floral odorants include jasmine, lilac, lily of the valley, magnolia, rose, lavender, geranium, hyacinth, orange blossom, apple blossom, carnation, and mixtures thereof. Examples of spice odorants include cinnamon, ginger, cloves, nutmeg, oriental spice, and mixtures thereof. In a preferred embodiment, a mixed floral odorant and a mixed spice odorant are employed. An exemplary floral and spice odorant mixture comprises mixed floral odorant IFF-31854-11 and mixed spice odorant IFF-91171 (oriental spicy) and/or IFF-91164 (floral semi-oriental spicy musk) from International Flavors and Fragrances, Inc., (IFF), New York, N.Y. Floral and spice odorants, and other odorants for use in the present methods, are readily available from a variety of commercial sources, including, for example, IFF, Energy Essentials, AromaTech, Inc. (Somerville, N.J.), Florasynth, Inc. (Teterboro, N.J.), among others, and as essential oils.

Odorants or odorant mixture can be readily screened and assessed for positive hedonics and effectiveness in altering the perception of body weight. For example, an odorant or odorant mixture can be administered to an individual who is questioned as to a positive or negative reaction to the pleasantness of the scent. The odorant or odorant mixture can then be administered to the individual for inhalation to assess its effectiveness in modifying their perception of body weight of another individual to a reduced body weight. The odorant or odorant mixture can also be assessed for its effect in providing a positive perception on the viewing individual (who inhales the odorant/mixture) of another individual. The odorant or odorant mixture can also be assessed for its effect on alleviating and reducing the anxieties of the other individual, which are associated with their negative feeling of being overweight, as a result of the positive effect of the odorant/mixture on the viewing individual.

For example, a method of screening an odorant or a mixture of odorants for effectively altering perception of body weight can comprise the steps of: administering a suprathreshold and non-irritant concentration of a composition consisting essentially of one or more odorants to an individual for inhalation; having the individual estimate the body weight of a person; comparing the estimate of the body weight to actual body weight of the person to provide a “difference value”; comparing the difference value to a “control value” to determine the statistical significance of the difference value; and eliminating the odorant or odorant mixture as being ineffective for altering perception of body weight if not statistically significant. The control value can be derived by having the person estimate the body weight of the individual without inhaling the composition (or inhaling an odorless control composition), and comparing the body weight estimate with the actual weight of the individual to provide the control value, preferably before administering the test composition to be screened.

The screening test as well as the method of the invention can include other steps such as having the person identify the composition as hedonically positive or hedonically negative, testing olfactory ability and/or olfactory capacity, among other olfactory tests known and used in the art.

According to the invention, the odorant or odorant mixture, e.g., mixed floral and spice odorants, is dispensed in an amount and time effective to provide a vaporous emission for inhalation by the individual to effectively change the individual's perception of another individual's body weight. Such an effect can be assessed and measured subjectively by interviewing and questioning the individual about their perception of body weight before and after the administration of the odorant or odorant mixture, and assessing their response.

In one embodiment, the method of the invention can be used to alter the perception of a second person (observer) of the body weight of an overweight individual, particularly an obese or morbidly obese individual, and thus increase the attractiveness and positive perception of the overweight/obese person to the observer. This can, in turn, have secondary effects on the overweight/obese person by enhancing a positive perception of themselves. In such a method, an effective concentration of a suitable odorant or odorant mixture is administered to the observing individual for inhalation for an effective time period such that they perceive the body weight of the overweight individual to be less than the actual body weight, preferably lower by at least about 7%, more preferably by at least about 10% or greater. In one embodiment of the method, an effective concentration of the odorant or odorant mixture is administered directly to the observing individual for inhalation, for example, by means of the odorant or odorant mixture applied to their body (e.g., skin) or clothing, or through the use of a dispensing device containing the odorant or odorant mixture (e.g., spray container, blister pack, cloth, etc.) which is presented to the observing individual. In another embodiment of the method, the odorant or odorant mixture is applied to the overweight individual such that a sufficient concentration of the odorant or odorant mixture can be inhaled by the observing individual to effect a change in their perception of the body weight of the overweight individual. The details of formulating and applying a suitable concentration of the odorant or mixed odorant composition to the overweight individual to achieve that end is well within the skill of the art.

In another embodiment, a suitable odorant or odorant mixture can be administered to an individual for inhalation to alter their perception of their own body weight, including body size, such that they perceive their own body weight to be less than actual body weight, preferably lower by at least about 7%, more preferably by at least about 10% or greater. The method can be utilized, for example, for treating a person who is identified as being overweight, particularly an individual identified as obese or morbidly obese. The medically defined threshold for being overweight (i.e., obese) is a body mass index (BMI) of 25 or greater. (National Heart, Lung, and Blood Institute, Body Mass Index Table: Obesity Guidelines (2001)). The method can also be used for treating a patient who is diagnosed as having an eating disorder, for example, bulimia nervosa, binge eating disorder, and anorexia nervosa. The method can be further applied to treating a patient who is diagnosed with a psychological disorder such as body dysmorphic disorder, social phobias, and pathological shyness, for example.

In addition to altering perception of body weight, administering the odorant or odorant mixture according to the invention can result in increasing the odorant-inhaling person's positive view of their own body weight, including body size, and improving their feeling of well being. The administration of a weight perception altering or “thinning” odorant/odorant mixture can be used to decrease anxiety felt by overweight individuals including obese and morbidly obese persons, individuals diagnosed with an eating disorder who perceive themselves to have a body weight that is higher than their actual weight, and/or individuals diagnosed with body dysmorphic disorder or other like psychological disorder, to help them to more easily assimilate into an everyday environment by changing their perception of their body weight. The method can also be used in connection with other treatments of individuals who are overweight or suffering from an eating or psychological disorder.

In another aspect, the method can utilize a food odorant used alone or as a combination of two or more food odorants. Food odorants refer to substances having a flavor quality (i.e., salty, sweet, sour, bitter) combined with an aromatic quality. The food odorant or mixture is a formulation that essentially comprises the effective food odorant or a blend of effective food odorants, and eliminates ineffective odorants that compete with the accords or notes of the effective food odorants to achieve a full effect on the individual inhaling the odorant or odorant mixture. Preferably, the food odorant or mixture provides a hedonically positive response by the inhaling individual. Examples of sweet food odorants include almond (benzaldehyde), anise, carob, cinnamon, cocoa (natural cocoa nibs), peanut (roasted), pecan, pistachio (natural), peppermint, spearmint, and fruit types including apple (natural apple), banana, fig, kiwi, orange (sweet orange peel) peach, raisin, and raspberry, among others. Examples of salty food odorants include artichoke, asparagus, avocado, basil, broccoli, celery seed, cucumber, fennel, garlic, horseradish, mushroom, mustard (seed), and spinach, pizza, white cheese (e.g., Romano), and yellow cheese (e.g., cheddar). Other useful food odorants can include vanilla, green apple, and chocolate. Food odorants can be a naturally-derived material or synthetically produced, according to methods known and used in the art, for example, by concentrating the flavor components (extracts) of naturally occurring plants and herbs, and parts thereof. Suitable food odorants for use in the present methods are readily available from a variety of commercial sources, including, for example, International Flavors and Fragrances, Inc. (IFF), New York, N.Y., Sensient Technologies Corporation, Gold Coast Ingredients, Inc., Flavor Concepts, and SpiceTec-USF, among others.

The odorant or odorant mixture is dispensed to the person in a form that provides a vaporous emission for inhalation. The odorant or odorant mixture can be administered to an individual, for example, by applying the odorant/odorant mixture to the individual's skin in a number of forms including, for example, as a liquid, powder, gel, cream, paste, and the like. The odorant or odorant mixture can be administered in combination with an odorless carrier such as mineral oil or water and/or odorless additives, and can be formulated with a viscosity effective to allow for aerosolization. The odorant composition can be dispensed onto the skin, for example, by direct application, by contact with a cloth carrying the composition, by an aerosol or nonaerosol spray, among other modes of application.

The odorant or odorant mixture can also be inhaled from a device, for example, from a capped vessel containing a liquid or solid form of the odorant or mixture, the liquid form being optionally absorbed to a wicking material, from a blister pack or scratch-and-sniff odor patch containing microcapsules of the odorant, as a spray from an aerosol or non-aerosol pump-type spray device, as a nasal spray, by means of a scented cloth, and the like. It is preferred that the odorant or odorant mixture is provided in a portable dispenser that is easily transportable and readily accessible.

The odorant or odorant mixture can be packaged as part of an article of manufacture, or kit. In one embodiment, the article of manufacture can comprise a container of an odorant or mixture of odorants or, packaged together, a container of a first odorant and a container of a second odorant (etc.) for combining together to form the odorant composition. The composition comprises a hedonically positive odorant or mixture of odorants in a suprathreshold and non-irritant concentration effective to alter perception of body weight upon inhalation such that the body weight is perceived as being less than the actual body weight. In a preferred embodiment, the composition consists essentially of a mixture of a floral odorant and a spice odorant. For example, the article of manufacture can comprise a container of a composition consisting essentially of a mixture of a floral odorant and a spice odorant or, packaged together, a container of a composition consisting essentially of a floral odorant and a container of a composition consisting essentially of a spice odorant. Examples of containers include a vial, jar, pouch, can, bottle, cloth, aerosolizer, blister pack, a booklet of scratch-and-sniff odor patches, and the like. The article of manufacture can further comprise written or other format of instructions (e.g., C.D., video, cassette tapes, etc.) for use of the composition for altering perception of body weight in a method according to the invention, including, but not limited to applications such as treating anxiety associated with obesity and improving body image, treating an eating disorder, etc. In another embodiment, the article of manufacture can comprise packaging material and an odorant composition according to the invention contained within the packaging material, wherein the packaging material comprises a label that indicates that the composition can be used for altering perception of body weight, treating a food disorder in a patient, treating a psychological disorder in a patient, and/or alleviating stress in a person. The article of manufacture can also include an odorant composition and instructions for testing olfactory threshold according methods known in the art. The parts of the article of manufacturing can be contained or separately packaged within a packaging material, such as a box, bag, pouch, etc.

An odorant is presented at a suprathreshold level when the decismel level or concentration of the odorant is beyond that needed to be detected by a normosmic individual. At its irritative level, the odorant quantity is so high and intense that the odorant stimulates predominantly the trigeminal nerve (for pain) rather than the olfactory nerve and, hence, is perceived as noxious or painful. The irritation threshold of the patient is the lowest concentration of the substance that causes immediate stinging or burning sensations in the nose, or stinging or lacrimation of the eye. (See, J. F. Gent, in Clinical Measurement of Taste and Smell, pages 107-166, H. L. Meiselman et al. (eds.), 602 pp., MacMillan, N.Y. (1986); R. L. Doty et al., Ann. Neurol. 25: 166-171 (1989); E. Koss et al., Neurology 38: 1228-1232 (1988); and R. Doty, The Smell Identification Test: Administration Manual 1983: 13-14, Philadelphia: Sensonics, Inc. (1983)).

If desired, prior to the administration of the odorant, the individual can undergo olfactory testing according to a test such as the University of Pennsylvania Smell Identification Test (UPSIT), a 40-question forced-choice, scratch-and-sniff identification test, and the Chicago Smell Test, a 3-item detection and identification test (R. Doty, The Smell Identification Test: Administration Manual 1983: 13-14, Philadelphia: Sensonics, Inc. (1983); A. R. Hirsch et al., Chemical Senses 18(5): 570-571 (1993); A. R. Hirsch et al., Chemical Senses 17(5): 643 (1992)).

The individual can also be evaluated for olfactory capacity (e.g. loss of smell) according to an olfactory threshold test as known and used in the art. Such a test provides a precise magnitude of loss of smell and classifies the individual as normosmic, hyposmic or anosmic, which is useful in assessing the effectiveness of a particular odorant and/or the required concentration of the odorant to provide a suprathreshold level to effectively change perception of body weight. According to that test, an odorant substance such as butyl alcohol, phenyl ethyl alcohol, or pyridine, is combined in a odorless liquid medium to provide a series of dilutions, or binary steps, of the odorant. For each successive binary step up the dilution scale, the odorant is present, for example, at one half the concentration of the preceding step. The highest concentration of the odorant usually provides the substance at an irritant level. The individual is presented with the series of dilutions in ascending order, and is asked to compare each dilution step to at least one control stimulus, such as odorless propylene glycol.

Ranges of the average normal threshold for various odorant substances can be found in the art, for example, Amoore and O'Neill, “Proposal for Unifying Scale to Express Olfactory Thresholds and Odor Levels: The “Decismel Scale”,” in Proceedings of the 1988 Air Pollution control Association Annual Meeting, Paper No. 78.5 (21 pp.), Air and Waste Management Association, Pittsburgh, Pa. (1988); Amoore and Haotala, “Odor as an Aid to Chemical Safety: Odor Thresholds Compared with Threshold Limit Values and Volatiles for 214 Industrial Chemicals in Air and Water Dilution,” J. Appl. Toxicology 3(6):272-290 (1983).

A suprathreshold amount is a concentration of the odorant/odorant mixture is greater than the threshold amount. The normal threshold concentration can be determined by administering a series of the same concentrations of the odorant/odorant mixture to a control group of at least 25 individuals who do not have a chemosensory dysfunction, and calculating the mean threshold concentration detected by the group of 25 individuals. Another alternative is to refer to the known threshold concentration value for the odorant/odorant mixture that has been established previously and published by J. Amoore et al., J. Appl. Toxicology, 3:272 (1983).

Odor thresholds can be expressed on the decismel scale. The decismel scale is constructed by setting the mean threshold concentration of a chemosensory agent detected by the control group of 20 year olds at the 0 value. A decismel is calculated by dividing the concentration of the odorant detected by the patient by the normal threshold concentration (using the published value or empirically determining the value) and then taking the logarithm of the quotient. The logarithm of the quotient is then multiplied by 20 to obtain the decismel value. Decismel values can be positive or negative. A positive decismel value indicates the patient is less sensitive to the odorant, i.e. has a higher threshold detection concentration. A negative decismel value indicates that the patient is more sensitive to the compound, i.e. has a lower threshold detection concentration. An increase in the threshold concentration value over the mean threshold concentration value of 2-fold corresponds to 6 decismels (or ds). Determination of decismel units is known in the art, as addressed, for example, in U.S. Pat. Nos. 5,380,765 and 5,492,934 (Hirsch).

In the art, a “normosmic” individual is one who can detect the odor of a substance without irritant sensations when the odorant is presented with the range of its average normal threshold. A “hyposmic” or “microsmic” individual has reduced capacity of the olfactory nerve being able to detect an odorant substance by its odor at a concentration, or decismel level, above that of a normosmic individual yet below its irritant concentration level. An “anosmic” individual is one who has essentially no olfactory nerve capacity being unable to detect the odor of the odorant substance, but has trigeminel nerve function, being able to detect an odorant substance by means of irritant, tingling sensations when it is present at an irritant concentration. A patient who is able to detect pyridine vapor by means of irritant, tingling sensations caused by stimulation of the trigeminel nerve, but who cannot distinguish a pyridine odor at a lower concentration without such sensation, is considered to be anosmic having no olfactory nerve sensitivity.

The invention will be further described by reference to the following detailed example. This example is not meant to limit the scope of the invention that has been set forth in the foregoing description. Variation within the concepts of the invention are apparent to those skilled in the art. The disclosures of the cited references throughout the application are incorporated by reference herein.

### EXAMPLE

Test Study. The following study was conducted to assess the effect of three odorant mixtures to induce a change in men's perception of body weight of a female individual.

A 5′9″, 245 lb., white, cosmetic saleswoman was blinded to the study hypothesis and served as the model for weight assessment. In order to avoid uncontrolled variables, she was instructed not to speak and to display minimal affect. She was instructed to wear the same clothing and to avoid change in hairstyle, makeup, and accessories for each day of the study. She was also to avoid use of fragranced items, including cologne, perfume, scented soap and shampoo.

One hundred ninety-nine (199) adult male volunteers, aged 12 to 61, were surveyed. Ninety-three percent (n=186) were white, 2 percent (n=4) were African American descent, 3 percent (n=6) were Hispanic descent, and 1.5 percent (n=3) were Asian descent.

Three odorant mixtures were judged to be hedonically positive by a panel at the Smell & Taste Treatment and Research Foundation, Chicago, Ill. Odorant #1 was a citrus and floral odorant mixture, Odorant #2 was a mixture of sweet pea and lily of the valley odorants, and Odorant #3 was a mixture of floral and spice odorants. For each day of the study, the odorants 1-3 were sequentially applied over separate days (i.e., one odorant per day for three consecutive days) to the model at non-irritant, suprathreshold levels. The testing was conducted indoors. The model stood at a distance that was predetermined to be adequate for normosmics to be able to detect the applied odorant at a non-irritant, suprathreshold level. The subjects were asked by one of the investigators to estimate the model's weight. To avoid a Hawthorne Effect whereby self-consciousness due to being observed can induce a change in a subjects' behavior (G. Adair, The Hawthorne Effect: A Reconsideration of the Methodological Artifact, Journal of Applied Psychiatry 69:2:334 345 (1984)), the protocol was designed with a written form such that the subjects wrote down the estimated weight without either the examiner or the model seeing the estimate.

Subjects were then queried on their detection and hedonics of each of the Odorants 1-3. On each day, estimates were performed by subjects without any odorant applied to the woman model in the control trials and with an Odorant 1-3 applied to the model in the experimental trials. Results were statistically analyzed independently for significance (p<0.05) by the University of Illinois School of Public Health. Comparisons were determined for each Odorant as compared to the control (no odorant) as well as subgroupings based on age and hedonics (i.e., like/dislike of the odorant mixture).

Since not all trials were performed in one day, to minimize fluctuations in the model's true weight, the tests were performed on consecutive days and non-odorant base line perceptions of weight were obtained for each trial day.

Pilot Study. In a pilot study conducted prior to the test study, a lavender odorant, pumpkin pie odorant, and cinnamon odorant, were individually applied in three different sessions to a woman subject who had a body mass index (BMI) of 23.0 as opposed to a BMI of 36.2 of the model in the test study. Despite the hedonically positive nature of the three odorants (i.e., lavender, pumpkin pie, cinnamon), none of the three odorants provided a weight-reducing effect on the perception of a group of male observers. The men judged the model not to weigh any less in the presence of the three odorants that were tested.

Results. The pilot study and the test study involved testing men's estimation of weight rather than women's on the basis that weight is a correlate of attractiveness in women, not men (D. M. Garner et al., Psychological Reports 47:483-491 (1980); M. Tovee et al., Proceedings of the Royal Society London (Series B): Biological Sciences 266:211-218 (1999); M. J. Tovee et al., Optimum Body-Mass Index and Maximum Sexual Attractiveness, Lancet 352:548 (1998)). Weight inversely correlates with the level of attractiveness more often when men look at women than when women look at men. Also, “societies value women's physical attractiveness more because physical attractiveness represents a more salient way of evaluating women's [social] role fulfillment than it does for men.” (D. BarTal and L. Saxe, Physical Attractiveness and its Relationship to Sex-Role Sterotyping, Sex Roles 2:123-133 (1976)).

The test study demonstrated that of the three odorant mixtures tested, only the use of a floral-spice odorant mixture had a specific effect and provided a perceived reduction of weight. In the test study, among the 50 males tested with the woman model wearing the odorant mixture of floral and spice odorants (Odorant #3), there was noted a significantly reduced perception of weight (p=0.02) compared to the control trials. This was a reduction of perceived weight by 4.1 pounds (a 2.4% reduction) as compared to the “no odor” control group in which the averaged perceived weight by 49 males was 172.8 pounds. Among the 100 males tested, neither the citrus-floral odorant mixture (Odorant #1) nor the sweet pea and lily of the valley odorant mixture (Odorant #2) were effective in reducing perception of weight (p=0.1, 0.01).

In those males who perceived Odorant #3 to be hedonically positive (n=16), the effect of the odorant on perceived weight of the model was even more substantial at 160.8 pounds (averaged), which was 12 pounds less than with “no odor” and 84 pounds less than the actual weight measurement (p=0.02). The inhalation by those test subjects of the floral-spice odorant mixture applied to the model resulted in a 7% reduction in perceived weight (p=0.02) compared to the no odor control subjects.

The results of the pilot study indicate that the effect of the odorant/odorant mixture can be less pronounced in cases in which the individual has a low body weight and is at a maximally attractive weight, such that the observer may or may not perceive the individual as weighing less, and/or relates a low body weight as being too thin and unattractive. Although Applicant does not intend to be bound by theory, it is believed that there is a body mass threshold at which the odorant/odorant mixture does not significantly alter the perception of body weight due to the body size of the individual. In the pilot study, the model was not judged to weigh any less in the presence of any of the three odorants. The model's BMI was 23.0 as opposed to the BMI of 36.2 of the model in the test study. It is believed that the model in the pilot study was already at a maximal attractive weight, and that the method of the invention is more effective to alter perception of the body weight of individuals whose BMI is greater than 25.0, the medically defined threshold for being overweight. (National Heart, Lung, and Blood Institute, Body Mass Index Table: Obesity Guidelines (2001)).

Discussion. The test study surprisingly showed that certain odorants have a significant impact on human perception of body weight of a woman. Certain odorants have also been found to significantly affect learning rate, insomnia, migraine headaches, aggression, and even gambling behavior.

Superficially, the floral-spice odorant mixture would not appear to provide a health benefit. However, this does not take into consideration the detrimental effects on psychological and social health of the perception of being overweight. When perceived as being overweight, women can avoid socialization and interpersonal contact. This often leads to a sedentary lifestyle, which further exacerbates a weight problem. By blocking negative image, an odorant or odorant mixture can act to break this vicious cycle and thus promote weight loss, enhance self-esteem, and healthy social relationships.

By inducing an effect by which an individual perceives herself as being perceived by others as being slimmer, it can induce a sense of attractiveness and thus self-confidence, which then can act to alleviate the impact of a variety of psychological conditions, not just anorexia or obesity but psychological disorders unrelated to weight such as social phobia and pathological shyness.

For individuals with body dysmorphic disorder and anorexia nervosa, where self-perception of the degree of obesity is markedly incongruent with reality, the use of a slimming odorant or mixture as a potential treatment approach is important. While many methods of treatment of bulimia nervosa and anorexia exist, none have been universally effective. Several treatments involve the reduction of anxiety. Vomiting in bulimia nervosa may be a form of self-treatment to reduce anxiety. Individuals with eating disorders have also been shown to be more likely to experience social phobias. By wearing a pleasant odorant or odorant mixture that effectively lowers another person's perception of their body weight, an individual with such disorders can experience an increase in positive self image, which can reduce both anxiety and facilitate approach behavior in social situations. Attaining an increased positive view of their own weight can allow them to divert their focus away from activities to reduce perceived weight and negative aspects of their body towards more healthy endeavors.

Similarly, obese and morbidly obese women often avoid social situations due to an embarrassment of their body size and lack of self-esteem. Many obese patients have been characterized with deficient self-esteem, strong fears of rejection linked with a compulsion to please others, lack of self-assertiveness, poor work histories, and body image distortions. The use of a thinning odorant or odorant mixture according to the invention, can break this downward cycle. Odorants can create a pleasant feeling towards others, facilitating the self-fulfilling prophecy that can influence the manner in which an individual is treated in social situations. In turn, the overweight individual may interpret this positive treatment as a sign of approval and reciprocate positive interaction with others. Therefore, use of a hedonically positive odorant may encourage them to socialize more and thus achieve a better quality of life.

The invention has been described by reference to detailed examples and methodologies. These examples are not meant to limit the scope of the invention. It should be understood that variations and modifications may be made while remaining within the spirit and scope of the invention, and the invention is not to be construed as limited to the specific embodiments described. The references and patents cited throughout the application are incorporated by reference herein.

What is claimed is:

1. A method of modifying perception of body weight, comprising the step of:
administering to a person for inhalation an effective amount of a composition comprising a hedonically positive mixture of a floral odorant and a spice odorant, wherein the person perceives the body weight to be about 5-10% less than actual body weight.

2. The method of claim 1, wherein the composition comprises a suprathreshold but non-irritant concentration of the odorants.

3. The method of claim 1, wherein the step of administering comprises dispensing the composition from a dispensing device.

4. The method of claim 1, wherein the step of administering comprises applying the composition onto the person.

5. A method of modifying perception of body weight of a person, comprising the step of:
administering to the person for inhalation a composition comprising a suprathreshold but non-irritant concentration of a hedonically positive mixture of a floral odorant and a spice odorant, wherein the person perceives the body weight to be about 5-10% less than actual body weight.

6. A method of altering perception of body weight comprising the step of:
administering a hedonically positive mixture of a floral odorant and a spice odorant to a person for inhalation such that the person perceives the body weight to be less than the actual body weight.

7. A method of altering perception of body weight, comprising the step of:
administering to a person for inhalation a hedonically positive mixture consisting essentially of a floral odorant and a spice odorant such that the person perceives the body weight to be less than the actual body weight.

8. A method of altering perception of body weight, comprising the step of:
administering a hedonically positive mixture of a floral odorant and a spice odorant to a first person for inhalation such that the first person perceives the body weight of a second person to be less than the actual body weight.

9. The method of claim 8, wherein the step of administering comprises applying an effective amount of the mixture of odorants onto the second person for inhalation by the first person.

10. The method of claim 8, wherein the step of administering comprises dispensing the mixture of odorants from a dispensing device to the first person for inhalation.

11. The method of claim 8, wherein the body mass index of the second person is 25 or greater.

12. An article of manufacture, comprising a container of a composition consisting essentially of a suprathreshold and non-irritant concentration of a mixture of a floral odorant and a spice odorant.

13. An article of manufacture, comprising: packaged together, a container of a composition consisting essentially of a suprathreshold and non-irritant concentration of a floral odorant, and a container of a composition consisting essentially of a suprathreshold and non-irritant concentration of a spice odorant.

14. An article of manufacture, comprising: packaging material and, contained within the packaging material, a suprathreshold and non-irritant concentration of a hedonically positive composition consisting essentially of a floral odorant and spice odorant, wherein the packaging material comprises a label that indicates that the composition can be used for altering perception of body weight.

15. An article of manufacture, comprising: packaging material and, contained within the packaging material, a suprathreshold and non-irritant concentration of a hedonically positive composition consisting essentially of a floral odorant and spice odorant, wherein the packaging material comprises a label that indicates that the composition can be used for altering perception of body weight, treating a food disorder in a patient, treating a psychological disorder in a patient, alleviating stress in a person, or a combination thereof.

16. The article of claim 15, wherein the psychological disorder is selected from the group consisting of body dysmorphic disorder, social phobia, and pathological shyness.

17. The article of claim 15, wherein the food disorder is selected from the group consisting of anorexia nervosa, bulimia nervosa, and binge eating disorder.

18. An article of manufacture, comprising, packaged together:
a container of a composition comprising a hedonically positive and suprathreshold and non-irritant concentration of one or more odorants; and
instructions for use of the composition for altering perception of body weight in a method comprising administering an effective amount of the composition to a person for inhaling.

19. The article of claim 18, wherein the composition consists essentially of a mixture of a floral odorant and a spice odorant.

20. A composition consisting essentially of a suprathreshold and non-irritant concentration of a floral odorant and a spice odorant, the composition being hedonically positive and effective to alter a person's perception of body weight upon inhalation whereby the body weight is perceived as being less than the actual body weight.

21. The composition of claim 20, wherein the floral odorant has the odorant characteristics of mixed floral odorant IFF # 31854-11, and the spice odorant has the odorant characteristics of oriental spicy odorant IFF# 91171 or floral semi-oriental spicy musk odorant IFF # 91164.

22. A method of screening an odorant or a mixture of odorants for effectively altering perception of body weight, comprising the steps of:
administering a suprathreshold and non-irritant concentration of a composition consisting essentially of one or more odorants to an individual for inhalation;
having the individual estimate the body weight of a person;
comparing the estimate of the body weight to actual body weight of the person to provide a difference value;
comparing the difference value to a control value to determine the statistical significance of the difference value; and
eliminating the odorant or odorant mixture as being ineffective for altering perception of body weight if not statistically significant.

23. The method of claim 22, wherein the control value is derived by having the person estimate the body weight of the individual without the composition administering step, and comparing the body weight estimate with the actual weight of the individual to provide the control value.

24. The method of claim 22, further comprising, prior to the composition administering step, the steps of having the person estimate the body weight of the individual, and comparing the body weight estimate with the actual weight of the individual to provide a control value.

25. The method of claim 22, further comprising the step of having the person identify the composition as hedonically positive or hedonically negative.
